(19) United States
(12) Patent Application Publication
(10) Pub. No.: US 2024/0331804 A1
(43) Pub. Date: Oct. 3, 2024

(54) INFORMATION PROCESSING DEVICE AND INFORMATION PROCESSING METHOD

(71) Applicant: Hitachi, Ltd., Tokyo (JP)

(72) Inventor: Tomohiro YASUDA, Tokyo (JP)

(21) Appl. No.: 18/595,044

(22) Filed: Mar. 4, 2024

(30) Foreign Application Priority Data
Mar. 28, 2023 (JP) 2023-051508

(51) Int. Cl. G16B 30/10; G16B 40/00

(52) U.S. Cl. CPC G16B 30/10; G16B 40/00

(57) ABSTRACT

Labeled positions alignment capable of dealing with apparent expansion and contraction of a target nucleic acid sequence is performed. An information processing device calculates first ratios of intervals between partial sequences in a reference nucleic acid sequence, constructs an index indicating a combination of the first ratios and information indicating a position of a partial sequence in the nucleic acid sequence corresponding to the combination of the first ratios, calculates second ratios of intervals between partial sequences in a target nucleic acid sequence, extracts a combination of the first ratios corresponding to a combination of the second ratios based on a comparison result between the combination of the second ratios and the combination of the first ratios indicated by the index, and outputs information indicating a position of a partial sequence corresponding to the extracted combination of the first ratios in the reference nucleic acid sequence.

### CLAIM OF PRIORITY

The present application claims priority from Japanese patent application JP 2023-051508 filed on Mar. 28, 2023, the content of which is hereby incorporated by reference into this application.

### BACKGROUND OF THE INVENTION

### 1. Field of the Invention

The present invention relates to an information processing device and an information processing method.

### 2. Description of Related Art

With the advancement of a deoxyribonucleic acid (DNA) sequencing technique, many personal genomes have been sequenced. A personal genome has many differences from a reference genome. Although most of differences in the personal genomes are single nucleotide variants (SNV) in which only one base in surrounding base sequences is different from that on the reference genome, the personal genomes also include structural variants (SV) in which a large sequence of thousands of bases or more changes at one time and the number of which is smaller than the number of SNVs.

SNV and SV include not only germline variants that cause individual differences, but also acquired variants called somatic variants, and some acquired variants can cause cancer. Accurately detecting such variants and elucidating biological significance and clinical significance of the variants are important tasks in cancer treatment and biological research.

In order to clarify a structural variant, it is necessary to capture changes in a large genome region of thousands of bases or more. However, a length of a base sequence that can be read at one time using a current DNA sequencing technique is limited. A length of a base sequence that can be read at one time is limited to a maximum of about 1000 bases in a Sanger method that is originally used to determine a standard human genome sequence, and is limited to about hundreds of bases in a current mainstream i.e. next generation sequencing (NGS).

In the NGS, a pair of two base sequences that are referred to as a paired end sequence and are separated from each other by about hundreds of bases can be obtained, and even when the paired end sequence is used, only a sequence of a narrow region in a range of about 1000 bases can be obtained. However, human genomes include many repetitive sequences such as short intersparsed nuclear elements (SINE) and long intersparsed nuclear elements (LINE), and repetitive sequences also in regions called centromeres and telomeres.

When only base sequences of 1000 bases at most are observed at one time, repetitive sequences cannot be distinguished from one another, and thus base sequences of an entire genome cannot be estimated when the obtained base sequences are assembled. Although a base sequence of tens of thousands of bases can be obtained at one time in long-read sequencing techniques which have been widely used in recent years, it is not sufficient to identify positions of repetitive sequences. Accordingly, a technique for analyzing a wide region on a genome is needed.

What can be used for such an application is a technique called genome mapping in which a specific short base sequence on a genome is labeled with fluorescence or the like, and a position on the genome is identified based on a pattern of a label interval. In the genome mapping, DNA constituting a genome is amplified and cut to generate a large number of DNA fragments including hundreds of thousands of bases.

In the genome mapping, a specific base sequence on each of a large number of generated DNA fragments is labeled, and a labeled position indicating about the number of bases between the beginning and each label sequence (hereinafter, also simply referred to as a label) is measured. Further, by arranging labeled positions on each DNA fragment in ascending order, the DNA fragment can be converted into a numerical value sequence in ascending order. This numerical value sequence is hereinafter also referred to as measurement data.

JP2009-022274A discloses such a genome mapping technique. JP2009-022274A discloses that “a method of mapping a position on a chromosomal DNA, including hybridizing a nucleic acid with one type of repeated base sequences in an expanded or elongated chromosomal DNA, measuring a mutual distance on the chromosomal DNA between a plurality of sets of repeated base sequences of the chromosomal DNA by using a label introduced into the hybridized nucleic acid, and then determining, based on a feature of the measured distance, a region or positions on chromosomes in the sets and the repeated base sequences included in the sets” (see abstract).

Alignment is defined as processing of comparing measurement data obtained by genome mapping with a labeled position obtained from a reference genome sequence or the like to clarify common portions or non-common portions. When a structural variant in a DNA fragment serving as a base of measurement data do not contain SVs or measurement errors, each labeled position indicated by the measurement data corresponds to a labeled position on a reference genome. On the other hand, when a structural variant occurs, corresponding positions between a label on the measurement data and a label on the reference genome are discontiguous. A structural variant can be detected by capturing such an abnormality in labeled positions.

### CITATION LIST

### Patent Literature

PTL 1: JP2009-022274A

### SUMMARY OF THE INVENTION

In order to detect a structural variant, it is required to accurately perform alignment. When there are a large number of errors at the time of alignment, an abnormality in labeled positions caused by the errors may be erroneously recognized as a structural variant.

In order to align measurement data, it is required to deal with errors included in the measurement data. As one of such errors, an entire length of each DNA fragment may appear to be apparently expanded or contracted in the measurement data. This is because moving speeds of molecules during measurement are not uniform.

JP2009-022274A discloses a method for labeling repeated sequences on a genome to identify positions on the genome, but does not disclose a method for collating a measured label interval with a labeled position of a reference genome.

On the other hand, according to an aspect of the invention, labeled positions alignment capable of dealing with apparent expansion and contraction of a target nucleic acid sequence is performed.

In order to solve the above problems, the following configuration is adopted in one aspect of the invention. An information processing device has a processor, and a memory. The memory stores first numerical value sequences indicating positions of a partial sequences in a reference nucleic acid sequence and a second numerical value sequence indicating measurement positions of the partial sequence in a target nucleic acid sequence. The processor is configured to calculate a plurality of first ratios of intervals between the partial sequences in the reference nucleic acid sequence based on the first numerical value sequence, construct an index indicating a combination of the first ratios and information indicating the position of the partial sequence in the reference nucleic acid sequence corresponding to the combination of the first ratios, calculate a plurality of second ratios of intervals between the partial sequences in the target nucleic acid sequence based on the second numerical value sequence, extract a combination of first ratios corresponding to a combination of the second ratios based on a comparison result between the combination of the second ratios and the combination of the first ratios indicated by the index, and output information indicating a position of the partial sequence corresponding to the extracted combination of the first ratios in the reference nucleic acid sequence.

According to one aspect of the invention, it is possible to perform labeled positions alignment capable of dealing with apparent expansion and contraction of a target nucleic acid sequence.

Problems, configurations, and effects other than those described above will become apparent in the following description of embodiments.

### BRIEF DESCRIPTION OF THE DRAWINGS

FIG. 1 is a block diagram showing a configuration example of a genome labeled position alignment device according to a first embodiment.

FIG. 2 is a diagram showing a data configuration example of measurement data according to the first embodiment.

FIG. 3 is a flowchart showing an example of index construction according to the first embodiment.

FIG. 4 is a diagram showing an outline example of the index construction according to the first embodiment.

FIG. 5 is a diagram showing a data configuration example of an index according to the first embodiment.

FIG. 6 is a flowchart showing an example of index search according to the first embodiment.

FIG. 7 is a flowchart showing an example of processing of searching for an index while skipping a part of labels according to the first embodiment.

FIG. 8 is a diagram showing an example of a DNA fragment in which labels are skipped in the measurement data according to the first embodiment.

FIG. 9 is a flowchart showing an example of alignment probability calculation processing according to the first embodiment.

FIG. 10 is a sequence diagram showing an example of overall processing executed by the genome labeled position alignment device.

FIG. 11 is a diagram showing an example of tree structures constructed by expanding k tuples indicated by an index according to a second embodiment.

FIG. 12 is a flowchart showing an example of alignment processing using the tree structures according to the second embodiment.

FIG. 13 is a flowchart showing an example of update processing of sets S and T executed in the alignment processing using the tree structures according to the second embodiment.

FIG. 14 is a diagram showing an example of a user interface displayed by an input and output device according to a third embodiment.

FIG. 15 is a diagram showing an example of structural variant detection processing according to a fourth embodiment.

### DESCRIPTION OF EMBODIMENTS

Hereinafter, a genome labeled position alignment device according to an embodiment of the invention will be described. The same reference signs are given to the same components in the following drawings, and repeated description will be omitted.

Errors included in measurement data include not only apparent expansion and contraction of a length of an entire DNA fragment described above, but also a detection failure of a label on the DNA fragment, erroneous detection at a position without a label on the DNA fragment, cut-off of the DNA fragment at the time of sample adjustment, and the like. The genome labeled position alignment device according to the embodiment executes alignment processing capable of dealing with such errors.

### First Embodiment

### Device Configuration

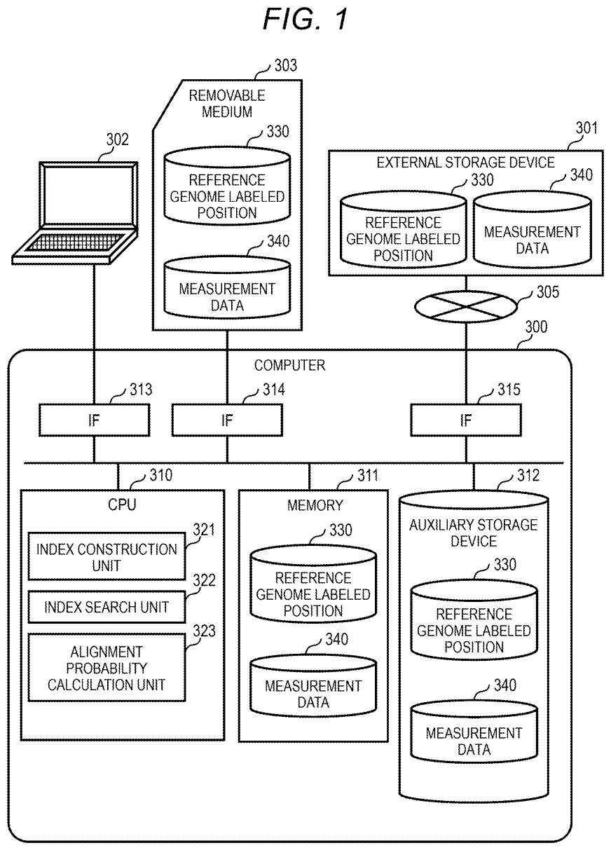

FIG. 1 is a block diagram showing a configuration example of a genome labeled position alignment device. The genome labeled position alignment device is implemented by, for example, a computer 300 including a central processing unit (CPU) 310, a memory 311, an auxiliary storage device 312, and interfaces 313 to 315. Hardware provided in the computer 300 is electrically connected via an internal communication line such as a bus.

The CPU 310 reads a program and data that are stored in the memory 311 and executes the program stored in the memory 311. The CPU 310 includes a processor. The CPU 310 includes, for example, an index construction unit 321, an index search unit 322, and an alignment probability calculation unit 323, which are functional units. The computer 300 functions as a genome labeled position alignment device when the CPU 310 executes processing.

The memory 311 temporarily stores a program to be executed by the CPU 310 and data used when the program is executed. The memory 311 includes a read only memory (ROM) which is a nonvolatile storage element and a random access memory (RAM) which is a volatile storage element. The ROM stores an invariable program (for example, basic input/output system (BIOS)) and the like. The RAM is a high-speed volatile storage element such as a dynamic random access memory (DRAM), and temporarily stores a program to be executed by the CPU 310 and data used when the program is executed.

The memory 311 stores, for example, a reference genome labeled position 330, measurement data 340, and programs for implementing the index construction unit 321, the index search unit 322, and the alignment probability calculation unit 323.

For example, the CPU 310 functions as the index construction unit 321 by being operated according to an index construction program loaded into the memory 311, functions as the index search unit 322 by being operated according to an index search program loaded into the memory 311, and functions as the alignment probability calculation unit 323 by being operated according to an alignment probability calculation program loaded into the memory 311.

The auxiliary storage device 312 stores a program to be executed by the CPU 310 and data used when the program is executed in a nonvolatile manner. That is, a program is read from the auxiliary storage device 312, loaded into the memory 311, and executed by the CPU 310.

The auxiliary storage device 312 is a large-capacity and nonvolatile storage device such as a hard disk drive (HDD) and a solid state drive (SSD). The auxiliary storage device 312 stores programs for implementing functions of the index construction unit 321, the index search unit 322, and the alignment probability calculation unit 323, the reference genome labeled position 330, and the measurement data 340.

The interfaces 313 to 315 convert a medium or a protocol of transmission and reception of a signal and are connected to an external device. The interface 313 is an I/O interface connected to an input and output device 302 in a wired manner or a wireless manner. The input and output device 302 includes an input device such as a keyboard and a mouse, and an output device such as a display device and a printer. The interface 313 acquires input information from an operator received by the input and output device 302. The interface 313 outputs an execution result of a program to the input and output device 302 in a format that can be visually recognized by the operator.

The interface 315 is a network interface connected to an external storage device 301 via a network 305. The interface 315 controls communication with other devices according to a predetermined protocol.

The external storage device 301 is a non-transitory storage device that stores data handled by the computer 300. The external storage device 301 includes, for example, a storage device such as an HDD or an SSD. The external storage device 301 can store the reference genome labeled position 330 and the measurement data 340.

Data transmission and reception between the external storage device 301 and the computer 300 are performed via the network 305.

The network 305 includes, for example, a local area network (LAN) and the Internet. A type of the network 305 is not limited to those described above. The network 305 may be implemented in a wired manner or a wireless manner.

The interface 314 is connected to a drive device that reads and writes a removable medium 303. The interface 314 includes, for example, a serial interface such as a universal serial bus (USB).

The removable medium 303 is a non-transitory storage medium that stores data handled by the computer 300. The removable medium 303 includes an optical disk such as a CD or a DVD, a magnetic disk, and a semiconductor memory. The removable medium 303 can store the reference genome labeled position 330 and the measurement data 340.

Some or all programs executed by the CPU 310 may be provided to the computer 300 from the removable medium 303 which is a non-transitory storage medium via the interface 314, or from the external storage device 301 which is a non-transitory storage device or from an external computer including the external storage device 301 via the network 305, and may be stored in the nonvolatile auxiliary storage device 312 which is a non-transitory storage medium.

Although the external storage device 301 and the removable medium 303 are connected to the computer 300 constituting the genome labeled position alignment device in FIG. 1, these external devices may be not provided when these devices are not necessary.

The input and output device 302 including the external storage device 301 may be connected to the computer 300 via the network 305. The computer 300 may be incorporated with a device having an input and output function, instead of the input and output device 302.

The index construction unit 321 constructs an index based on k tuples. The index construction unit 321 constructs an index using k tuples based on a ratio of label intervals which will be described later, instead of an index using k tuples indicating an interval of labeled positions (hereinafter also simply referred to as a label interval). The index constructed by the index construction unit 321 can deal with a case where a part of labels is missing so as to deal with a label detection failure.

The k tuples indicate a combination of k numerical values (k is a parameter defined in advance). As in the related art, it is assumed that k tuples are a combination of k label intervals on a reference genome, and an index is constructed. The index is a correspondence table between the k tuples and identifiers of labels on the reference genome corresponding to label intervals in the k tuples.

In this case, apparent expansion and contraction of the measurement data 340 cannot be dealt with simply by generating the k tuples in the measurement data 340 and performing alignment by comparing the generated k tuples with k tuples indicated by an index.

In order to deal with the apparent expansion and contraction of the measurement data 340, when k tuples are generated by apparently expanding and contracting the entire measurement data 340 at various expansion and contraction ratios, and the k tuples corresponding to the expansion and contraction are compared with k tuples indicated by the index, since it is necessary to try out a large number of potentially correct expansion and contraction ratios in order to improve alignment accuracy and adopt an optimal expansion and contraction ratio, processing time may be increased.

Even when a huge variety of types of expansion and contraction ratios are adopted, the all adopted expansion and contraction ratios may deviate from an expansion and contraction ratio that is optimal for the measurement data 340, and as a result, it is not possible to perform accurate alignment.

At the time of measuring a DNA fragment, since moving speeds of DNA fragment molecules are not uniform at the time of measurement, label intervals in the measurement data 340 greatly expand and contract, but an error of a ratio of label intervals in the measurement data 340 is small. Therefore, in the embodiment, the index construction unit 321 constructs an index using k tuples based on a ratio of label intervals as described above, and thus it is possible to perform highly accurate alignment that does not require expansion or contraction of the measurement data 340.

The index search unit 322 uses the index based on the k tuples constructed by the index construction unit 321 to identify a position on a reference genome that is highly likely to correspond to the measurement data. The index search unit 322 executes processing of corresponding to erroneous detection of a label at the time of searching.

The alignment probability calculation unit 323 calculates a probability indicating occurrence of generated alignment. In a case where there are a plurality of candidates for positions where the DNA fragment indicated by the measurement data 340 is aligned, since probability indicating accurate alignment is high, the alignment probability calculation unit 323 adopts alignment having a high probability. Even when only a part of the DNA fragment can be aligned, the probability is used to determine whether the alignment is optimal.

Before starting genome labeled position alignment processing, the reference genome labeled position 330 and the measurement data 340 are input and stored in the computer 300. For example, the CPU 310 may read the reference genome labeled position 330 and the measurement data 340 and load the reference genome labeled position 330 and the measurement data 340 into the memory 311 when the computer 300 is started or when the computer 300 executes processing.

The reference genome labeled position 330 and the measurement data 340 may be stored in all of the auxiliary storage device 312, the external storage device 301, and the removable medium 303, or may be stored in some of the auxiliary storage device 312, the external storage device 301, and the removable medium 303. The data may be moved or copied to the external storage device 301 or the removable medium 303 when the computer 300 is stopped or when an available space of the auxiliary storage device 312 is insufficient. Therefore, it is desirable that the reference genome labeled position 330 stored in different storage devices is the same information. The same applies to the measurement data 340.

The reference genome labeled position 330 includes numerical data indicating positions of labels present on each of a plurality of reference genomes. The measurement data 340 includes data obtained by measuring a labeled position on each of a large number of DNA fragments.

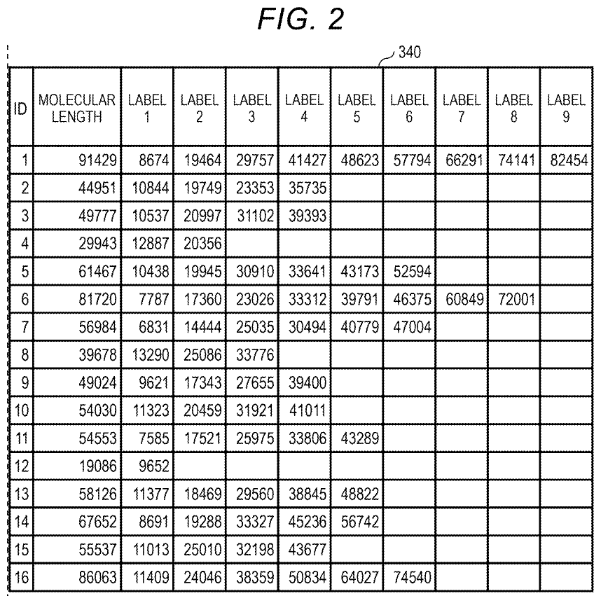

FIG. 2 is a diagram showing a data configuration example of the measurement data 340. The measurement data 340 indicates an ID for identifying a DNA fragment (an example of a target nucleic acid sequence), a molecular length of a DNA fragment (a length of a base sequence), and a position of a label on a DNA fragment (an example of a partial sequence). A blank in the measurement data 340 indicates that no label is observed.

For example, a molecular length of a DNA fragment with an ID of “2” is 44951 bases, four labels are measured in the DNA fragment, and positions of the four labels are a 10844th base, a 19749th base, a 23353rd base, and a 35735th base from the beginning of the DNA fragment. A position of a label on a DNA fragment indicated by the measurement data 340 may indicate, for example, a beginning position or an ending position of the label. In the embodiment, GCTCTTC recognized by an enzyme called Nt.BspQI serves as an example of a short base sequence to be labeled on a DNA fragment. As described above, the measurement data 340 indicates labeled positions of GCTCTTC in DNA fragments in ascending order. That is, the measurement data 340 indicates information in which labeled positions of DNA fragments are recorded as a numerical value sequence in ascending order (an example of a second numerical value sequence).

### Index Construction

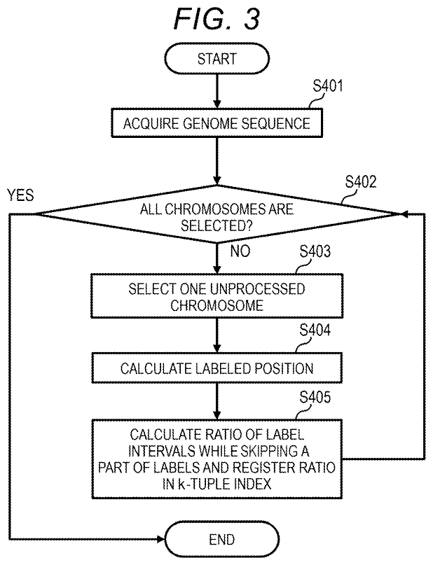

FIG. 3 is a flowchart showing an example of index construction processing. The index construction unit 321 constructs k tuples based on a ratio of label intervals for any one labeled position on a reference genome, and constructs an index indicating a correspondence relationship between the constructed k tuples and a labeled position on the reference genome.

Step S401: the index construction unit 321 acquires genome sequence data. The genome sequence data indicates a number of one of a plurality of chromosomes which are elements of a genome, and a base sequence (an example of a reference nucleic acid sequence) of each of the plurality of chromosomes. The base sequence of each of the plurality of chromosomes is represented by a character string including characters A, T, G, and C representing four types of bases and N representing an unknown base. The genome sequence data is stored in advance in, for example, at least one of the memory 311, the auxiliary storage device 312, the removable medium 303, and the external storage device 301.

Step S402: when the index construction unit 321 determines that all chromosomes has been selected, the index construction processing is stopped, and when the index construction unit 321 determines that there is an unselected chromosome, the processing proceeds to step S403.

Step S403: the index construction unit 321 selects one unprocessed chromosome. The index construction unit 321 constructs k tuples and registers the k tuples in an index for the selected chromosome with the following steps.

Step S404: the index construction unit 321 calculates a numerical value sequence (an example of a first numerical value sequence) indicating a position of a label (an example of a partial sequence) on the chromosome selected in the most recent step S403, and stores the numerical value sequence and a number of the chromosome in the reference genome labeled position 330 in association with each other. For example, the above-described GCTCTTC is used as a label sequence. Since the genome DNA is a double helix, it should be noted that a site where a complementary sequence (a sequence in which A, T, G, C are replaced with T, A, C, G in reverse order) matches with the label sequence is also labeled in genome mapping. Accordingly, when the index construction unit 321 calculates a numerical value sequence indicating a labeled position, the index construction unit 321 needs to add a position corresponding to the label sequence or the complementary sequence of the label sequence to the numerical value sequence without distinction.

Step S405: based on the numerical value sequence indicating a labeled position calculated in step S404, the index construction unit 321 calculates a ratio of label intervals and a ratio of label intervals when a part of labels is skipped, registers the k tuples in the index based on the calculated ratios, and returns the processing to step S402.

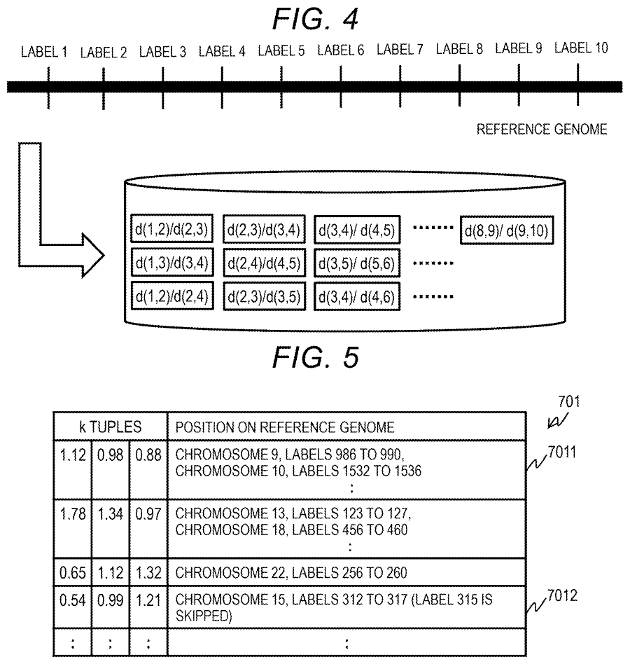

A specific example of the processing in step S405 will be described with reference to FIG. 4. FIG. 4 is a diagram showing an outline example of the index construction processing. In the example in FIG. 4, it is assumed that 10 labels consisting of labels 1 to 10 are present on the reference genome, and labeled positions of the labels 1 to 10 are obtained. An interval between a label i and a label j is denoted by d (i, j).

In step S405, first, the index construction unit 321 calculates, using the numerical value sequence indicating a labeled position calculated in step S404, a label interval between adjacent labels and a label interval between labels adjacent to each other when a part of labels are skipped according to a predetermined rule. Here, the predetermined rule is, for example, a rule for skipping a label when the k tuples are generated using k ratios of label intervals, and any one of a second label to a (k+2)-th label is skipped in contiguous (k+3) labels. That is, a target to be skipped is a label other than a first label and a (k+3)-th label at both ends. Hereinafter, an example of calculating the k tuples while sequentially skipping any one of the labels 1 to 10 will be described.

In this case, the index construction unit 321 calculates a label interval d (i, i+1) for i=1, . . . 9. Here, a label interval d (x, y) represents a distance between a label x and a label y, and a unit is the number of bases. Further, for example, since the label 1 and the label 3 are adjacent to each other by further skipping the label 2, the index construction unit 321 calculates a label interval between labels adjacent to each other by sequentially skipping a label according to the predetermined rule so as to further calculate a label interval d (1, 3) or the like.

The index construction unit 321 calculates a ratio of adjacent label intervals and a ratio of label intervals adjacent to each other by skipping a part of labels according to the predetermined rule. That is, the index construction unit 321 calculates d (i, i+1)/d (i, i+2) for i=1, . . . 8. Further, for example, since the label interval d (1, 3) and a label interval d (3, 4) are adjacent to each other by skipping the label 2, the index construction unit 321 calculates a ratio of label intervals adjacent to each other by sequentially skipping a label according to the predetermined rule so as to further calculate a label interval ratio d (1, 3)/d (3, 4) or the like.

The index construction unit 321 registers k tuples based on a combination of values of contiguous k ratios and positions on the reference genome corresponding to the k tuples in association with each other in an index based on the calculated ratios of label intervals. Further, the index construction unit 321 registers the k tuples based on a combination of values of k ratios that are contiguous when a part of labels are skipped according to the predetermined rule and the positions on the reference genome corresponding to the k tuples in association with each other in the index based on the calculated ratios of label intervals.

For example, a chromosome number from which the k tuples are calculated and an integer value indicating the number of k+2 labels serially assigned from the beginning of the chromosome from which a ratio value included in the k tuples is calculated are registered in the index as positions on the reference genome.

For example, in a case where k=3, for each of i=1, . . . 6, k tuples including d (i, i+1)/d (i+1, i+2), d (i+1, i+2)/d (i+2, i+3), and d (i+2, i+3)/d (i+3, i+4) are obtained as k tuples when no label is skipped for a number of a selected chromosome and a label i to a label i+4 which are positions on the reference genome.

Further, for example, the index construction unit 321 registers, in the index, k ratios of label intervals that are adjacent to each other by sequentially skipping labels according to the predetermined rule so as to register k tuples including d (1, 3)/d (3, 4), d (3, 4)/d (4, 5), and d (4, 5)/d (5, 6) in the index as k tuples for five labels of the label 1, the label 3, the label 4, the label 5, and the label 6 that are contiguous by skipping the label 2 of the selected chromosome, or the like.

When the index construction unit 321 registers a ratio value in an index, the index construction unit 321 may perform an operation of forcibly regarding a third decimal place or lower as zero and regarding close values as the same value using, for example, a method such as binning so as to absorb an error of the measurement data 340 that inevitably includes an experimental error.

In a case where the index construction unit 321 calculates a ratio between a first label interval and a second label interval adjacent to each other, when the first label interval is greatly larger than the second label interval, a ratio value greatly exceeds 1.0, and on the other hand, when the first label interval is smaller than the second label interval, a ratio value is smaller than 1.0, and thus non-symmetry depending on a magnitude relationship occurs. In order to prevent the non-symmetry, the index construction unit 321 may use a ratio logarithm (for example, a logarithm having a predetermined value as a base, such as a common logarithm or a natural logarithm), instead of a ratio value.

Since the k tuples express k ratios of label intervals, the index construction unit 321 may express a ratio of adjacent label intervals by an integer value such that a sum of the k ratio values matches an integer value given as a parameter, instead of directly calculating a ratio of adjacent label intervals. For example, in a case where an integer value given as a parameter is 100 and k=3, when three ratios of adjacent label intervals are 3000, 3000, and 4000, the k tuples may be expressed as integers having a sum of 100 such as 30:30:40.

FIG. 5 is a diagram showing a data configuration example of the index constructed in step S405. An index 701 indicates, for example, a correspondence between k tuples and positions on the reference genome corresponding to the k tuples. The index 701 is stored in, for example, the same storage device as the reference genome labeled position 330.

For example, a record 7011 indicates that all of k tuples in “labels 986 to 990” of a “chromosome 9”, k tuples in “labels 1532 to 1536” of a “chromosome 10”, . . . are “1.12, 0.98, 0.88”. For example, a record 7012 indicates that k tuples in “labels 312 to 317” (that is, a label 312, a label 313, a label 314, a label 316, and a label 317) when a “label 315” of a “chromosome 15” is skipped are “0.54, 0.99, 1.21”.

### Index Search

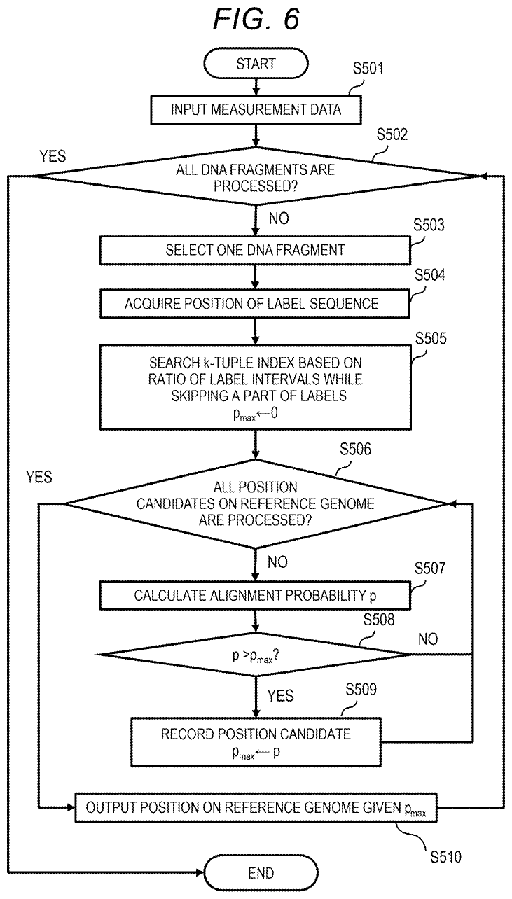

FIG. 6 is a flowchart showing an example of index search processing. The index search unit 322 constructs k tuples based on a ratio of label intervals on any position of a DNA fragment indicated by the measurement data 340, and identifies a position on the reference genome corresponding to the DNA fragment indicated by the measurement data 340 using the index 701 created by the index construction unit 321.

Step S501: the index search unit 322 inputs the measurement data 340.

Step S502: when the index search unit 322 determines that all DNA fragments included in the input measurement data 340 are selected, the index search unit 322 ends the index search processing, and when the index search unit 322 determines that there is an unselected DNA fragment, the index search unit 322 proceeds the processing to step S503.

Step S503: the index search unit 322 selects one unselected DNA fragment from the measurement data 340. The index search unit 322 may select a DNA fragment in any order, and for example, the index search unit 322 simply selects a DNA fragment in an order in which information is input to the measurement data 340.

Step S504: the index search unit 322 acquires, from the measurement data 340, detected labeled positions on the DNA fragment selected in the most recent step S503.

Step S505: the index search unit 322 calculates label intervals on the DNA fragment based on the labeled positions acquired in step S504, and calculates a ratio of adjacent label intervals. Then, the index search unit 322 compares k tuples which are values of contiguous k ratios on the DNA fragment with k tuples in the index constructed in step S321, and acquires a corresponding position candidate on the reference genome.

In step S505, the index search unit 322 further skips a label from the selected DNA according to the predetermined rule, compares k tuples generated when the skipped label is not present with the k tuples in the index 701, and acquires a corresponding position candidate on the reference genome so as to deal with erroneous detection of a label in the measurement data 340.

Accordingly, in step S505, a correspondence between labeled positions indicated by the k tuples on the selected DNA fragment and the position candidate on the reference genome is generated.

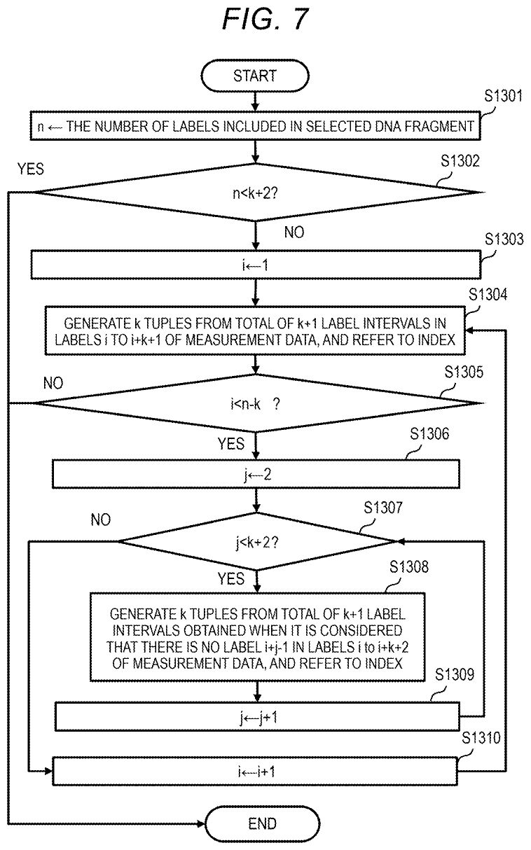

Details of index referring processing in step S505 will be described later with reference to FIG. 7. Further, the index search unit 322 initializes a variable pₘₐₓ to 0 in step S505.

Since a plurality of position candidates on the reference genome corresponding to the k tuples on the DNA fragment may appear, the position candidates are sequentially processed by the following procedure.

Step S506: when the index search unit 322 determines that all corresponding position candidates on the reference genome acquired in step S505 are processed, the index search unit 322 proceeds the processing to step S511, and when the index search unit 322 determines that there is an unprocessed position candidate, the index search unit 322 proceeds the processing to step S507.

Step S507: the alignment probability calculation unit 323 calculates, for each position candidate on the reference genome, a probability p that labeled positions indicated by the k tuples of the DNA fragment corresponding to the candidate are aligned.

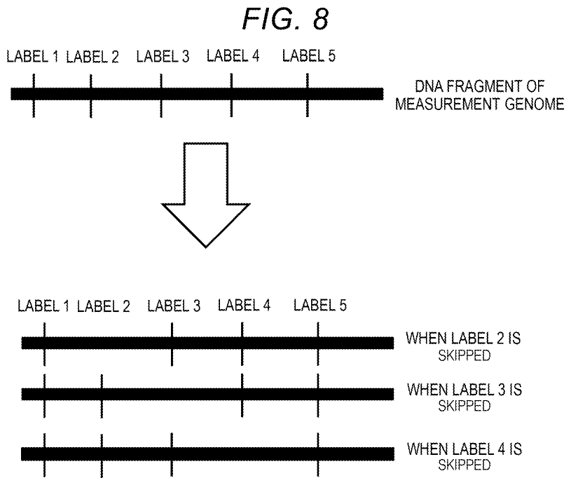

Details of a procedure for calculating the alignment probability p will be described later with reference to FIG. 8.

Step S508: when the alignment probability calculation unit 323 determines that p>pₘₐₓ, the alignment probability calculation unit 323 proceeds the processing to step S509, and when the alignment probability calculation unit 323 determines that p pₘₐₓ, the alignment probability calculation unit 323 returns the processing to step S506.

Step S509: the alignment probability calculation unit 323 records the selected position candidate, substitutes p for pₘₐₓ, and returns the processing to step S506. When a position candidate is already recorded, the alignment probability calculation unit 323 overwrites the position candidate.

Step S510: the index search unit 322 outputs the recorded position candidate, and ends the index search processing.

A method of outputting a position on the reference genome having a smallest probability is described in the procedure from steps S506 to S508. Alternatively, a procedure of outputting not only one position having a largest probability but also a plurality of candidates may be added.

FIG. 7 is a flowchart showing an example of processing of searching for the index 701 while skipping a part of labels in step S505. In the example shown in FIG. 7, the predetermined rule for skipping a part of labels is sequentially skipping any one label of labels included in the selected DNA fragment.

Step S1301: the index search unit 322 sets a variable n to the number of labels observed in the DNA fragment selected in the measurement data 340.

Step S1302: when n<k+2, the number of label intervals in the DNA is k or less and a ratio of the label intervals is k−1 or less, and thus the index search unit 322 cannot refer to the index 701 and ends the processing in FIG. 7. When n≥k+2, the index search unit 322 proceeds the processing to step S1303.

Step S1303: the index search unit 322 initializes a variable i to 1.

Step S1304: the index search unit 322 generates k tuples by calculating a total of k ratios from a total of k+1 label intervals among labels i to i+k+1 without skipping a label from the selected DNA fragment, and refers to the index 701. For example, in the processing of referring to the index 701, the index search unit 322 acquires k tuples matching with the generated k tuples from the index 701, and acquires positions on the reference genome corresponding to the acquired k tuples in the index 701 as candidates.

Step S1305: in a case where i≥n−k−1, the index search unit 322 ends the processing in FIG. 7 since the number of label intervals is k+1 or less when a label is skipped. In a case where i<n−k−1, the index search unit 322 proceeds the processing to step S1306.

Step S1306: the index search unit 322 initializes a variable j to 2. The reason why the variable j is not initialized to 1 is that a label at a left end does not need to be skipped (new k tuples are not generated even when the label at the left end is skipped).

Step S1307: when j≥k+2, the index search unit 322 proceeds the processing to step S1310. When j<k+2, the index search unit 322 proceeds the processing to step S1308.

Step S1308: the index search unit 322 generates k tuples by calculating a total of k ratios from a total of k+1 label intervals obtained when it is considered that there is no label i+j−1 in labels i to i+k+2 on the selected DNA fragment, and refers to the index 701. That is, the index search unit 322 refers to the index 701 using k tuples obtained by skipping the label i+j−1.

Step S1309: the index search unit 322 adds 1 to the variable j to update a label to be skipped to a subsequent label, and returns the processing to step S1307.

Step S1310: the index search unit 322 adds 1 to the variable i to update positions of the k tuples on the DNA fragment selected with reference to the index 701, and returns the processing to step S1304.

Index registration processing of the reference genome in step S405 can also be achieved by the same processing as the method shown in FIG. 7. Specifically, the “DNA fragment” in the processing shown in FIG. 7 may be replaced with a “chromosome”, and the processing of referring to the index 701 in FIG. 7 may be replaced with processing of registering the generated k tuples and the positions on the reference genome in the index 701 in association with each other.

FIG. 8 is a diagram showing an example of a DNA fragment in which a label is skipped in the measurement data 340. In the example shown in FIG. 8, it is assumed that five labels of labels 1 to 5 are observed in a DNA fragment in the measurement data 340. In a case where k=2, k tuples when no label is skipped from the DNA fragment, k tuples when the label 2 is skipped from the DNA fragment, k tuples when the label 3 is skipped from the DNA fragment, and k tuples when the label 4 is skipped from the DNA fragment are generated in the processing shown in FIG. 7.

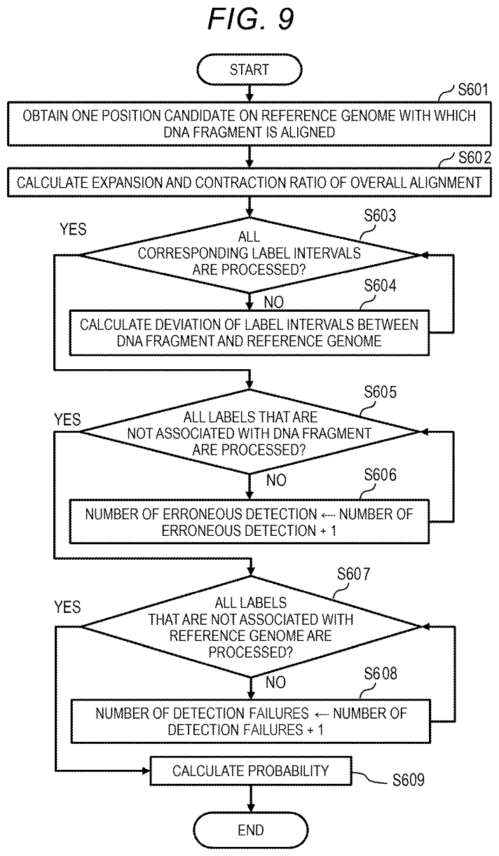

Alignment Probability Calculation FIG. 9 is a flowchart showing an example of alignment probability calculation processing in step S507. Hereinafter, m is an aligned portion of a DNA fragment to be subjected to a probability calculation, and P(m) is a probability indicating occurrence of the alignment (p in steps S507 to S509).

In the embodiment, for example, the alignment probability calculation unit 323 calculates P(m) using a probability model expressed by P(m)=P_{scale}^{w1}·Pₚₒₛ^{w2}·Pᵢₙₛ^{w3}·P_{del}^{w4}. P_{scale}, Pₚₒₛ, Pᵢₙₛ, and P_{del} are respectively a probability of an expansion and contraction ratio indicating a ratio of an observed molecular length of a DNA fragment to a molecular length of the reference genome, a probability of a deviation of an observed label interval (from an interval on a genome), a probability of erroneous detection of a label, and a probability of label detection failure. Further, w1, w2, w3, and w4 are weights, and may be set to w1=w2=w3=w4=1 unless otherwise specified by a user.

Although the probability model is expressed by P (m)=P_{scale}^{w1}·Pₚₒₛ^{w2}·Pᵢₙₛ^{w3}·P_{del}^{w4} in the example described above, some of P_{scale}^{w1}, Pₚₒₛ^{w2}, Pᵢₙₛ^{w3}, and P_{del}^{w4} may not be considered (that is, some of w1, w2, w3, and w4 may be 0).

The alignment probability calculation unit 323 calculates P_{scale} according to, for example, P_{scale}=f_{scale}(|expansion and contraction ratio−1|). For example, f_{scale} (x) is a probability density function of a normal distribution having an average of 0 and a variance σ_{scale}², and σ_{scale} is determined based on actual data given in advance.

The alignment probability calculation unit 323 calculates Pₚₒₛ according to, for example, Pₚₒₛ=fₚₒₛ (|x₁−y₁|) fₚₒₛ(|x₂−y₂|) . . . fₚₒₛ (|xₙ−yₙ|). For example, fₚₒₛ (x) is a probability density function of a normal distribution N (0, σₚₒₛ²), and σₚₒₛ² is determined based on actual data given in advance. Further, x₁ is an i-th label interval in the DNA fragment, and y₁ is a corresponding i-th label interval of a chromosome indicated by the reference genome.

The alignment probability calculation unit 323 calculates Pins using, for example, a function that monotonically decreases with an increase in nᵢₙₛ based on the number of times nᵢₙₛ of erroneous detection of a label. Specifically, for example, the alignment probability calculation unit 323 can calculate Pins according to Pᵢₙₛ(nᵢₙₛ)=Rᵢₙₛexp (−nᵢₙₛ) using an exponential function exp(x). For example, Rᵢₙₛ is a constant coefficient and is determined based on actual data given in advance.

Similarly, the alignment probability calculation unit 323 can calculate P_{del} according to P_{del} (n_{del})=R_{del}exp (−n_{del}) using, for example, the number of times n_{del} of label detection failures. Processing of calculating occurrence probability of alignment based on the above definition will be described.

Step S601: the alignment probability calculation unit 323 acquires a correspondence relationship between candidates of an alignment target obtained as a result of the referring processing in step S505, that is, labeled positions on the selected DNA fragment and labeled positions on the reference genome. That is, for example, the alignment probability calculation unit 323 specifies a portion where k tuples of the selected DNA fragment and k tuples of the reference genome indicated by the index 701 match with each other.

Step S602: the alignment probability calculation unit 323 calculates an expansion and contraction ratio of the entire alignment. Specifically, for example, the alignment probability calculation unit 323 can calculate an expansion and contraction ratio of the entire alignment by calculating a distance between labeled positions at both ends for each DNA fragment selected in step S503 and the reference genome (a chromosome) indicated by the correspondence relationship acquired in step S601 and by obtaining a ratio of the calculated distances between the labeled positions at both ends.

Step S603: the alignment probability calculation unit 323 calculates, in a loop of step S604, deviations |x₁−y₁| between all label intervals of the DNA fragments and label intervals of the reference genome that are indicated by the correspondence acquired in step S601. In step S603, the alignment probability calculation unit 323 determines whether all label intervals are processed.

When all label intervals are processed, the alignment probability calculation unit 323 proceeds the processing to step S605, and when there is an unprocessed label interval, the alignment probability calculation unit 323 proceeds the processing to step S604.

Step S604: the alignment probability calculation unit 323 calculates a deviation x₁−y₁| of label intervals for an associated pair of label intervals x₁ and y₁, and returns the processing to step S603.

Step S605: in a loop of step S606, the alignment probability calculation unit 323 counts the number of erroneous detection of labels on a DNA fragment assuming that labeled positions on the DNA fragment and positions on the reference genome that are indicated by the correspondence relationship acquired in step S601 match with each other. Here, the erroneous detection refers to a labeled position on the DNA fragment that does not correspond to a position on the reference genome. The alignment probability calculation unit 323 proceeds the processing to step S607 when all labels of the measurement data that do not correspond to those on the reference genome are processed, and proceeds the processing to step S606 when there is an unprocessed label of the measurement data that does not correspond to that on the reference genome.

Step S606: the alignment probability calculation unit 323 increments the number of erroneous detection by 1 and returns the processing to step S605. The number of erroneous detection is initialized to 0 in advance.

Step S607: in a loop of step S608, the alignment probability calculation unit 323 counts the number of detection failures of labels on the DNA fragment assuming that the labeled positions on the DNA fragment and the positions on the reference genome that are indicated by the correspondence relationship acquired in step S601 match with each other. Here, a detection failure refers to a labeled position on a reference genome that does not correspond to a label on the DNA fragment. The alignment probability calculation unit 323 proceeds the processing to step S609 when all labels on the reference genome that do not correspond to those in the measurement data are processed, and proceeds the processing to step S608 when there is an unprocessed label among labels on the reference genome that do not correspond to those in the measurement data.

Step S608: the alignment probability calculation unit 323 increments the number of detection failures by 1 and returns the processing to step S607. The number of detection failures is initialized to 0 in advance.

Step S609: the alignment probability calculation unit 323 calculates P(m) by substituting the numerical values obtained by the above processing (the expansion and contraction ratio obtained in step S602, the deviation of label intervals obtained in step S604, the number of erroneous detection obtained in step S606, and the number of detection failures obtained in step S608) into a definition formula of P(m), and ends the alignment probability calculation processing.

A parameter determined based on actual data is not a common value in the entire genome, and is preferably set individually for regions of each chromosome. According to the above processing, the genome labeled position alignment device according to the embodiment can align k tuples based on a ratio of label intervals on each DNA indicated by the measurement data 340 with k tuples based on a ratio of label intervals on the reference genome.

As described above, the genome labeled position alignment device according to the embodiment can perform alignment capable of dealing with expansion and contraction of a DNA fragment at the time of measurement by performing the alignment using k tuples based on a ratio of label intervals.

Further, the genome labeled position alignment device can perform alignment capable of dealing with erroneous detection of a label on a DNA fragment and a detection failure of a label on a DNA fragment by registering k tuples constructed while skipping a part of labels on a chromosome of a reference genome in the index 701 and comparing k tuples generated while skipping a part of a label on a DNA fragment indicated by the measurement data 340 with k tuples indicated by the index 701.

In the above processing, since the genome labeled position alignment device determines a corresponding position on the reference genome based on alignment probabilities using P_{scale}, Pₚₒₛ, Pᵢₙₛ, and P_{del}, the genome labeled position alignment device can perform alignment capable of dealing with expansion and contraction of a DNA fragment during measurement, a deviation of labeled positions on the DNA fragment, erroneous detection of a label on the DNA fragment, and a detection failure of a label on the DNA fragment. In addition, the genome labeled position alignment device can provide a method for performing alignment by evaluating reliability of partial alignment caused by DNA molecule cut-off or a structural variant at the time of sample adjustment.

### Second Embodiment

The genome labeled position alignment device can align k tuples based on a ratio of label intervals between the DNA fragment of the measurement data 340 and the reference genome in the first embodiment, while the genome labeled position alignment device expands alignment by sequentially associating labels with one another around the k tuples registered in the index 701 in the embodiment.

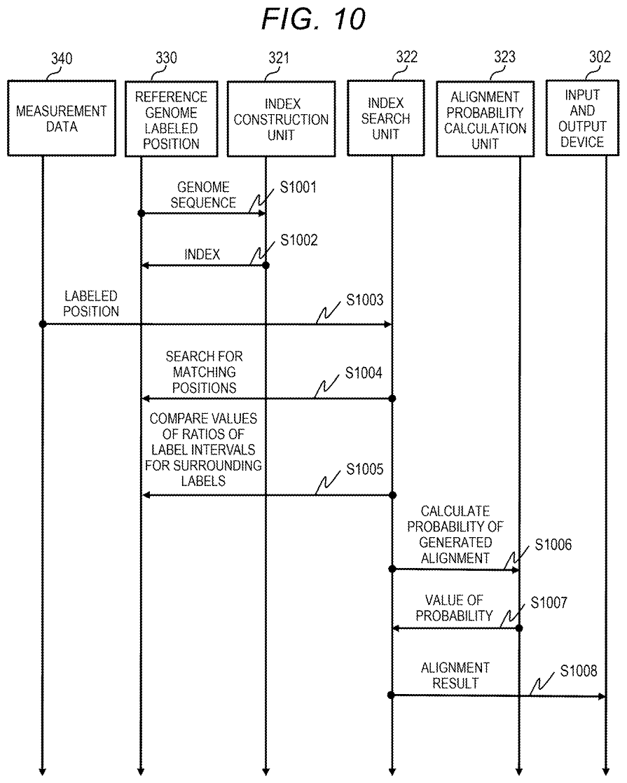

FIG. 10 is a sequence diagram showing an example of overall processing executed by the genome labeled position alignment device. In the processing shown in FIG. 10, a process in which the index construction unit 321, the index search unit 322, and the alignment probability calculation unit 323 perform cooperative operations will be described with reference to the reference genome labeled position 330 and the measurement data 340. According to the processing shown in FIG. 10, when there is no DNA molecule cut-off or structural variant at the time of sample adjustment, it is expected that all molecules indicated by the measurement data 340 can be aligned with the reference genome. The processing up to step S1004 are the same as those in the first embodiment.

Step S1001: the index construction unit 321 receives a genome sequence as an input.

Step S1002: the index construction unit 321 constructs the index 701 using k tuples based on a ratio of label intervals in steps S401 to S405.

Step S1003: the index search unit 322 acquires the measurement data 340 and acquires a labeled position of the DNA fragment included in the acquired measurement data 340.

Step S1004: the index search unit 322 refers to the index 701 and searches for positions on the reference genome with which the k tuples match while skipping a part of labels of the DNA fragment in steps S501 to S505.

Step S1005: the index search unit 322 compares a value of a ratio of label intervals on the DNA fragment with a value of a ratio of label intervals on the reference genome for labels around the labeled positions with which the k tuples match in step S1004.

Step S1006: the alignment probability calculation unit 323 calculates an alignment probability reflecting a comparison result of the surrounding label intervals in step S1005.

Step S1007: the alignment probability calculation unit 323 outputs the alignment probability calculated in step S1006 to the index search unit 322.

Step S1008: the index search unit 322 determines optimal alignment based on the alignment probability and outputs the determined alignment to the input and output device 302.

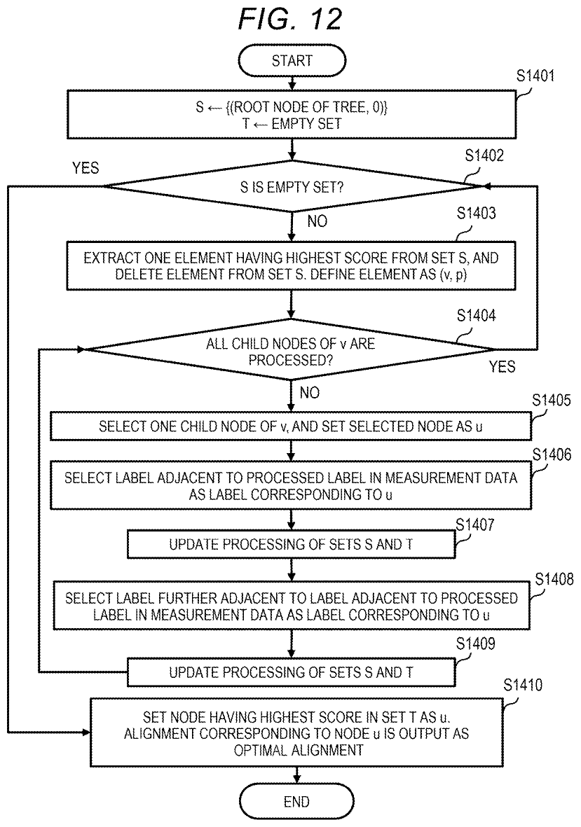

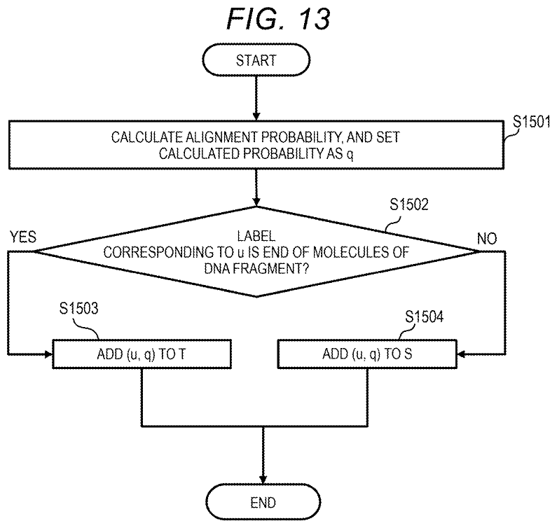

Details of steps S1005 to S1008 will be described later with reference to FIGS. 12 and 13.

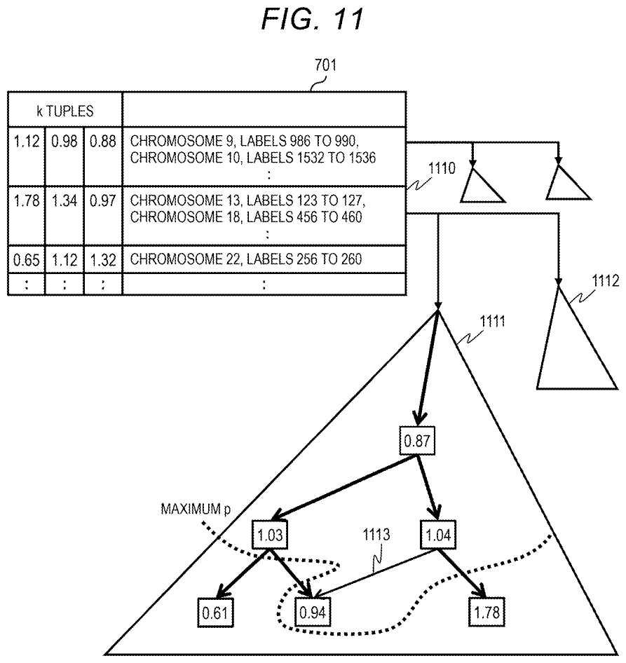

FIG. 11 is a diagram showing an example of tree structures constructed by expanding the index 701 in order to compare a ratio of label intervals around k tuples. In the embodiment, the index 701 is expanded, and the index construction unit 321 constructs two tree structures for each set of k tuples.

One tree structure corresponding to each set of k tuples indicates a ratio of label intervals upstream (a side close to the beginning of a character string expressing a chromosome) of the k tuples in the reference genome, and the other tree structure corresponding to the k tuples indicates a ratio of label intervals downstream (a side close to an ending position of the character string expressing the chromosome) of the k tuples.

In each tree structure, a node closest to a root indicates a ratio of label intervals adjacent to the k tuples, and a child node indicates a ratio of label intervals adjacent to a parent node. That is, in the case of a tree structure indicating a ratio of upstream label intervals, a ratio of upstream adjacent label intervals is shown, and in the case of a tree structure indicating a ratio of downstream label intervals, a ratio of downstream adjacent label intervals is shown.

Ratio values indicated by the k tuples and the tree structures are considered to be the same as values close to those using a method such as binning. Therefore, the same k tuples may appear at a plurality of locations on the reference genome, and since ratios of adjacent label intervals are different in the locations, one parent node may have a plurality of child nodes in the tree structures. This is why a data structure indicating a ratio of label intervals around label intervals indicated by the k tuples is a tree structure.

The index 701 and tree structures 1111 and 1112 constructed from the labeled position 330 on the reference genome by the index construction unit 321 in the example shown in FIG. 11 will be specifically described. A record 1110 of the index 701 shows k tuples in which a combination of ratios of adjacent three label intervals is “1.78”, “1.34”, and “0.97”, and positions on the reference genome corresponding to the k tuples. The tree structure 1111 indicates ratios of label intervals upstream of the k tuples indicated by the record 1110, and the tree structure 1112 indicates ratios of label intervals downstream of the k tuples indicated by the record 1110.

For example, for positions on the reference genome corresponding to the k tuples indicated by the record 1110, the tree structure 1111 is constructed by sequentially searching for a ratio of label intervals adjacent to “1.78” on an upstream side, and the tree structure 1112 is constructed by sequentially searching for a ratio of label intervals adjacent to “0.97” on a downstream side.

In the tree structure 1111, a node closest to a root node is “0.87”. This indicates that, a ratio of label intervals adjacent to “1.78” on the upstream side is “0.87” for all positions on the reference genome corresponding to the k tuples indicated by the record 1110.

Further, a node indicating “1.03” and a node indicating “1.04” serve as child nodes of “0.87” in the tree structure 1111. Accordingly, among positions on the reference genome corresponding to the k tuples indicated by the record 1110, a ratio of label intervals that are two intervals upstream of “1.78” (that is, a ratio of label intervals adjacent to “0.87” on the upstream side) is “1.03” or “1.04”.

Similarly, a node indicating “0.61” and a node indicating “0.94” are obtained as child nodes of “1.03”, and a node indicating “1.78” is obtained as a child node of “1.04” (a node indicating “0.94” is not obtained as a child node of “1.04”), by calculating a ratio of label intervals upstream of the positions on the reference genome.

Here, in a case including a node group having the same parent node as that in the tree structure 1111 and having close values (for example, a difference is within a predetermined value) such as “1.03” and “1.04”, for example, an edge 1113 from a node included in the node group to a child node of another node included in the node group may be added, or search processing shown in FIG. 12 to be described later may be executed assuming that such an edge is virtually present. By adding the edge 1113, even when a minute error is included in a calculated ratio of label intervals, it is highly likely to obtain correct alignment by searching of tracing the edge 1113.

The genome labeled position alignment device according to the embodiment compares a ratio of label intervals on a DNA fragment indicated by the measurement data 340 and a ratio of label intervals on the reference genome around the k tuples aligned by the procedure in the first embodiment using the above-described tree structures shown in FIG. 11, and generates optimal alignment. A method will be described below.

In order to guarantee that alignment is optimum, the genome labeled position alignment device can obtain final optimal alignment by sequentially calculating generation probabilities of alignment by the alignment probability calculation unit 323 and sequentially examining nodes capable of generating optimal alignment at that time, that is, alignment that maximizes the probability p calculated by the alignment probability calculation unit 323. More precisely, the calculation is performed by procedures shown in FIGS. 12 and 13.

FIG. 12 is a flowchart showing an example of alignment processing using tree structures.

Step S1401: the index search unit 322 initializes a set S with {(root node of tree, 0)}. The index search unit 322 initializes a set T to an empty set. A first value (a left-side element) included in each element of the set S is referred to as a node, and a second value (a right-side element) is referred to as a score. Hereinafter, calculation is performed such that the set S is a set of nodes in the middle of a search and the set T is a set of nodes corresponding to labels for which the search is completed.

Step S1402: the index search unit 322 proceeds the processing to step S1410 when the set S is an empty set, and proceeds the processing to step S1403 when the set S is not an empty set.

Step S1403: the index search unit 322 extracts one element having a highest score from elements of the set S and deletes the extracted element from the set S. The extracted element is defined as (v, p).

Step S1404: the index search unit 322 returns the processing to step S1402 when all child nodes of v are processed, and proceeds the processing to step S1405 when there is an unprocessed child node of v.

Step S1405: the index search unit 322 selects one child node of v and sets the selected node as u.

Step S1406: the index search unit 322 selects a label adjacent to the processed label (a label corresponding to v) in the measurement data 340 as a label corresponding to u.

Step S1407: the index search unit 322 and the alignment probability calculation unit 323 execute update processing of the sets S and T on u and the label selected in step S1406. Details of the update processing of the sets S and T will be described later with reference to FIG. 13.

Step S1408: the index search unit 322 selects a label further adjacent to the label corresponding to u in order to deal with a case where the label corresponding to u is not detected in a DNA fragment being processed.

Step S1409: the index search unit 322 and the alignment probability calculation unit 323 regard the label selected in step S1408 as a new u, execute the update processing of the sets S and T to be described later with reference to FIG. 13, and return the processing to step S1404 after the execution of the update processing.

Step S1410: the index search unit 322 sets a node having a highest score in the set T as u. The index search unit 322 outputs, as optimal alignment, alignment corresponding to the node u, that is, alignment in which a label on a genome corresponding to nodes selected from a tree structure to the final node u is associated with a label of the DNA fragment.

FIG. 13 is a flowchart showing an example of update processing of the sets S and T executed in the alignment processing using the tree structure.

Step S1501: the alignment probability calculation unit 323 calculates a probability of alignment corresponding to the selected node u using the method shown in FIG. 9 as in step S507, and sets the calculated probability as q.

Step S1502: the index search unit 322 determines whether a labeled position corresponding to the node u is an end of molecules of the DNA fragment. The index search unit 322 proceeds the processing to step S1503 when the index search unit 322 determines that the labeled position is an end, and proceeds the processing to step S1504 when the index search unit 322 determines that the labeled position is not an end.

Step S1503: the index search unit 322 adds (u, q) to the set T.

Step S1504: the index search unit 322 adds (u, q) to the set S.

### Third Embodiment

The third embodiment provides a user interface for setting a parameter for adjusting processing executed by the genome labeled position alignment device. The user interface visualizes an alignment result.

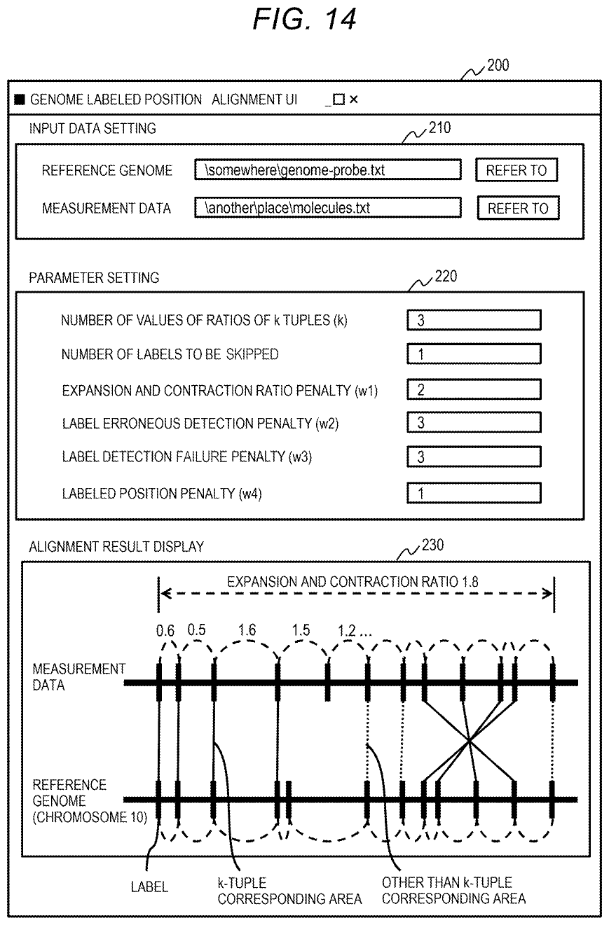

FIG. 14 is a diagram showing an example of a user interface displayed by the input and output device 302. The user interface 200 includes, for example, an input data setting area 210, a parameter setting area 220, and an alignment result display area 230.

The input data setting area 210 is an area for setting an acquisition source of the reference genome labeled position 330 and the measurement data 340. Although the acquisition source is designated by a file name in the example shown in FIG. 14, the acquisition source may be designated by a uniform resource locator (URL) or the like as necessary.

The parameter setting area 220 is an area for setting a parameter used for processing executed by the genome labeled position alignment device. For example, the number (k) of ratio values in k tuples can be set in the parameter setting area 220.

For example, the number of labels to be skipped by the index construction unit 321 and the index search unit 322 in order to deal with a detection failure and erroneous detection can be set in the parameter setting area 220. A case where the number of labels to be skipped is one is described in the example described above. Alternatively, the number of labels to be skipped may be set to two or more. When the number of labels to be skipped increases, a data size of the index 701 increases while error tolerance increases.

For example, parameters w1, w2, w3, and w4 for weighting errors used when the alignment probability calculation unit 323 calculates the probability P(m) can be set in the parameter setting area 220. Since the weights can be set in the parameter setting area 220, importance of various errors can be tuned.

Information for visualizing alignment obtained as a calculation result is displayed in the alignment result display area 230. A position on the reference genome corresponding to the DNA fragment of the measurement data 340 is displayed in the alignment result display area 230, and corresponding labels between the DNA fragment and the reference genome can be checked.

In the alignment result display area 230, it is also possible to distinguish between labels associated with k tuples (an example of a non-expanded portion, and k-tuple areas associated with solid lines in FIG. 14) and a peripheral area associated using a tree structure (an example of an expanded portion, and k-tuple areas associated with dotted lines in FIG. 14) among corresponding labels on the DNA fragment and the reference genome. In the alignment result display area 230, it is possible to assist a user in verifying a calculation result by showing a value of a ratio of label intervals and an expansion and contraction ratio of all molecules of the DNA fragment.

### Fourth Embodiment

In the embodiment, a genome labeled position alignment system detects a structural variant present in a genome of a subject. The genome labeled position alignment system includes a genome mapping device and the genome labeled position alignment device described in the first to third embodiments.

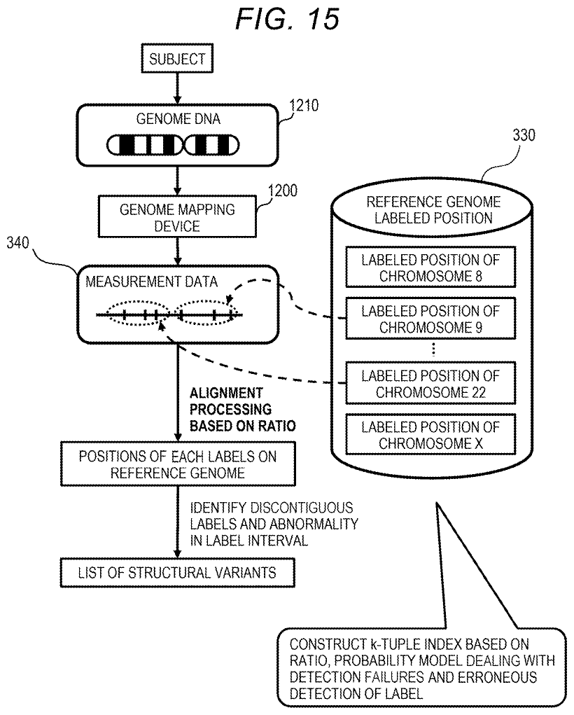

FIG. 15 is a diagram showing an example of structural variant detection processing. A genome mapping device 1200 acquires a genome DNA collected from a subject. The genome mapping device 1200 amplifies and fragments the genome of the acquired genome DNA of the subject. Further, the genome mapping device 1200 obtains the measurement data 340 by measuring positions of labels on DNA fragments.

The genome labeled position alignment device can identify positions on the reference genome corresponding to labels indicated by the measurement data 340 by aligning the DNA fragment indicated by the measurement data 340 with the reference genome labeled position 330 by alignment processing based on k tuples using a ratio of label intervals described in the first and second embodiments.

The genome labeled position alignment device determines whether a structural variant is present in the genome of the subject based on a comparison result between a labeled position on the DNA fragment indicated by the measurement data 340 and a labeled position on the reference genome corresponding to the labeled position on the DNA fragment.

Specifically, for example, when the genome labeled position alignment device determines that labeled positions on the genome of the subject are discontiguous (specifically, when labels on the reference genome corresponding to contiguous labels on the DNA fragment are discontiguous) or when the genome labeled position alignment device determines that a label interval is abnormally large or small (for example, a difference between a label interval on the DNA fragment indicated by the measurement data 340 and a label interval on the reference genome corresponding to the label interval on the DNA fragment is equal to or more than a predetermined value or less than a predetermined value), the genome labeled position alignment device can determine that a structural variant is present in the genome of the subject. The genome labeled position alignment device outputs a list of structural variants detected in such a manner to, for example, the input and output device 302, so that a user of the genome labeled position alignment device can comprehensively grasp structural variants present in the genome of the subject.

The invention is not limited to the above-described embodiments, and includes various modifications. For example, the embodiments described above have been described in detail to facilitate understanding of the invention, and the invention is not necessarily limited to those including all the configurations described above. A part of a configuration according to one embodiment can be replaced with a configuration according to another embodiment, and a configuration according to one embodiment can also be added to a configuration according to another embodiment. A part of a configuration according to an embodiment may be added, deleted, or replaced with another configuration.

Some or all of configurations, functions, processing units, processing methods, and the like described above may be implemented by hardware by, for example, designing with an integrated circuit. In addition, the above configurations, functions, and the like may be implemented by software by a processor interpreting and executing a program for implementing each function. Information such as a program, a table, and a file for implementing a function can be stored in a recording device such as a memory, a hard disk, or a solid state drive (SSD), or in a recording medium such as an IC card, an SD card, or a DVD.

Control lines and information lines indicate what is considered to be necessary for explanation, and not necessarily all control lines and information lines are always shown on a product. Actually, almost all components may be considered to be connected to one another.

What is claimed is:

1. An information processing device comprising:
a processor; and
a memory, wherein
the memory stores a first numerical value sequence indicating a position of a partial sequence in a reference nucleic acid sequence and a second numerical value sequence indicating a measurement position of the partial sequence in a target nucleic acid sequence, and
the processor is configured to
calculate a plurality of first ratios of intervals between the partial sequences in the reference nucleic acid sequence based on the first numerical value sequence,
construct an index indicating a combination of the first ratios and information indicating the position of the partial sequence in the reference nucleic acid sequence corresponding to the combination of the first ratios,
calculate a plurality of second ratios of intervals between the partial sequences in the target nucleic acid sequence based on the second numerical value sequence,
extract a combination of first ratios corresponding to a combination of the second ratios based on a comparison result between the combination of the second ratios and the combination of the first ratios indicated by the index, and
output information indicating a position of the partial sequence corresponding to the extracted combination of the first ratios in the reference nucleic acid sequence.

2. The information processing device according to claim 1, wherein
the processor is configured to calculate a ratio of intervals between the partial sequences adjacent to each other in the reference nucleic acid sequence and a ratio of intervals between the partial sequences adjacent to each other when a part of the partial sequences is skipped from the reference nucleic acid sequence based on a predetermined rule as the plurality of first ratios based on the first numerical value sequence.

3. The information processing device according to claim 1, wherein
the processor is configured to calculate a ratio of intervals between the partial sequences adjacent to each other in the target nucleic acid sequence and a ratio of intervals between the partial sequences adjacent to each other when a part of the partial sequences is skipped from the target nucleic acid sequence based on a predetermined rule as the plurality of second ratios based on the second numerical value sequence.

4. The information processing device according to claim 1, wherein
the processor is configured to
specify a combination of the first ratios matching with the combination of the second ratios from the index,
calculate, for the specified combination of the first ratios, a probability that the specified combination of the first ratios corresponds to the combination of the second ratios based on a predetermined probability model, and
extract a combination of the first ratios corresponding to the combination of the second ratios based on the calculated probability.

5. The information processing device according to claim 4, wherein
the predetermined probability model is a model that reflects at least one of a probability of an expansion and contraction ratio indicating a ratio of an observed molecular length to a correct molecular length of the target nucleic acid sequence, a probability of a deviation between a measurement position and a correct position of the partial sequence in the target nucleic acid sequence, a probability of erroneous detection of the partial sequence in the target nucleic acid sequence, and a probability of a detection failure of the partial sequence in the target nucleic acid sequence.

6. The information processing device according to claim 1, wherein
the processor is configured to
expand the combination of the first ratios based on a ratio of intervals between the partial sequences adjacent to a partial sequence in the reference nucleic acid sequence corresponding to the index for the combination of the first ratios indicated by the index,
expand the combination of the second ratios based on a ratio of intervals between the partial sequences adjacent to a partial sequence in the target nucleic acid sequence corresponding to the combination of the second ratios, and
extract the combination of the first ratios corresponding to the combination of the second ratios based on a comparison result between the expanded combination of the second ratios and the expanded combination of the first ratios.

7. The information processing device according to claim 6, wherein
the processor is configured to
specify the expanded combination of the first ratios matching with the expanded combination of the second ratios, and
output information indicating a position of a partial sequence of the reference nucleic acid sequence, which is indicated by the first ratio matching with a second ratio in an expanded portion of the expanded combination of the first ratios matching with the expanded combination of the second ratios, and information indicating a position of a partial sequence of the reference nucleic acid sequence, which is indicated by the first ratio matching with a second ratio in a non-expanded portion of the expanded combination of the first ratios matching with the expanded combination of the second ratios.

8. The information processing device according to claim 1, wherein
the information processing device is connected to an input device, and
the processor receives an input of the number of the first ratios included in the combination of the first ratios and the number of the second ratios included in the combination of the second ratios via the input device.

9. The information processing device according to claim 1, wherein
the processor is configured to
determine whether a structural variant is present in the target nucleic acid sequence based on a comparison result between a measurement position of a partial sequence in the target nucleic acid sequence indicated by the combination of the second ratios and a position of a partial sequence in the reference nucleic acid sequence indicated by the extracted combination of the first ratios, and
output information indicating the structural variant when it is determined that the structural variant is present in the target nucleic acid sequence.

10. An information processing method to be executed by an information processing device, the information processing device including a processor and a memory, the memory storing a first numerical value sequence indicating a position of a partial sequence in a reference nucleic acid sequence and a second numerical value sequence indicating a measurement position of the partial sequence in a target nucleic acid sequence, the information processing method comprising:
calculating, by the processor, a plurality of first ratios of intervals between the partial sequences in the reference nucleic acid sequence based on the first numerical value sequence;
constructing, by the processor, an index indicating a combination of the first ratios and information indicating the position of the partial sequence in the reference nucleic acid sequence corresponding to the combination of the first ratios;
calculating, by the processor, a plurality of second ratios of intervals between the partial sequences in the target nucleic acid sequence based on the second numerical value sequence;
extracting, by the processor, a combination of first ratios corresponding to a combination of the second ratios based on a comparison result between the combination of the second ratios and the combination of the first ratios indicated by the index; and
outputting, by the processor, information indicating a position of the partial sequence corresponding to the extracted combination of the first ratios in the reference nucleic acid sequence.
